(19) United States
(12) Patent Application Publication
(10) Pub. No.: US 2021/0052151 A1
(43) Pub. Date: Feb. 25, 2021

(54) DISPENSING DEVICE

(71) Applicant: BATRIK Medical Manufacturing Inc., Dorval (CA)

(72) Inventor: Mitchell K. GOLDBERG, Westmount (CA)

(21) Appl. No.: 16/999,543

(22) Filed: Aug. 21, 2020

Related U.S. Application Data
(60) Provisional application No. 62/889,602, filed on Aug. 21, 2019.

(51) Int. Cl. A61B 1/12; A61B 1/00; A61B 1/04

(52) U.S. Cl. CPC A61B 1/127; A61B 1/04; A61B 1/00087

(57) ABSTRACT

A dispensing device for dispensing anti-fog solution to a surface of medical device, the dispensing device comprising a container portion for storing the anti-fog solution to be dispensed; an applicator portion comprising a backing and an applicator attached to the backing and defining a port within the backing for fluid connection between the container portion and the applicator; a seal which is arranged to be selectively modulated from a closed configuration in which the anti-fog solution is maintained in the container portion, and an open configuration in which the anti-fog solution can flow through the port into the applicator portion; and a fluid flow modulator for controlling the flow of the anti-fog solution from the container portion to the applicator portion.

### FIELD OF THE DISCLOSURE

The present disclosure relates to a dispensing device, and more specifically although not exclusively, to a dispensing device for dispensing liquid such as anti-fog solution to a medical device surface.

### BACKGROUND OF THE DISCLOSURE

There are many types of dispensing devices for liquids. Anti-fog solution for medical device surfaces for which clear visibility of the surface is required is an example of a liquid requiring dispensing. Endoscope medical device lenses and mirrors are medical device surfaces which are prone to condensation and subsequent reduced visibility when close to or inside a body cavity of a patient due to temperature and humidity differences between the body cavity and the endoscope. Such reduced visibility can negatively impact the diagnostic effect of the medical device, for example.

Anti-fog solutions applied to the medical device surface can reduce the risk of condensation. The anti-fog solutions have compositions that typically include a surfactant for reducing surface tension and controlling droplet formation on the surface. Conventionally, anti-fog solutions are provided in a sterile pack comprising a single-use bottle with a lid and containing the anti-fog solution accompanied by a cloth or sponge for applying the solution to the medical device lens. A medical practitioner typically opens the sterile pack at the time of a medical procedure with the patient, and applies the anti-fog solution to the cloth or sponge and rubs it on the endoscope lens.

However, this is a cumbersome process requiring both hands of the medical practitioner and having a risk of any one or more of the separable items of the pack being accidentally left within a body cavity of the patient.

Therefore, there is a need for dispensing devices which overcome or reduce at least some of the above-described problems.

### SUMMARY OF THE DISCLOSURE

Broadly, there is provided a dispensing device of a one-piece construction that can allow the application of solution to a medical device surface single-handedly.

From one aspect, there is provided a dispensing device for dispensing anti-fog solution to a surface of medical device, the dispensing device comprising: a container portion for storing the anti-fog solution to be dispensed; an applicator portion comprising a backing and an applicator attached to the backing and defining a port within the backing for fluid connection between the container portion and the applicator; a seal which is arranged to be selectively modulated from a closed configuration in which the anti-fog solution is maintained in the container portion, and an open configuration in which the anti-fog solution can flow through the port into the applicator portion; and a fluid flow modulator for controlling the flow of the anti-fog solution from the container portion to the applicator portion.

In certain embodiments, the fluid flow modulator is a surface area reduction factor at the port compared to the container portion of about 1.1 to about 14.6, or about 3 to about 13, or about 4.

In certain embodiments, the port has cross-sectional surface area of about 2 mm² and the container portion has a cross-sectional surface area of about 12.6 mm².

In certain embodiments, the applicator surface area is about 390 mm².

In certain embodiments, the volume of anti-fog solution in the container option is about 6 ml.

In certain embodiments, the fluid flow modulator comprises a volume ratio of volume of the anti-fog solution in the container portion to a volume of the applicator of between about 400 mm³/ml and about 1000 mm³/ml.

In certain embodiments, the seal is arranged to be modulated from the closed configuration to the open configuration by application of a pressure to one or both of the container portion and the applicator portion.

In certain embodiments, the seal comprises a material which does not react with the anti-fog solution when in the closed position.

In certain embodiments, the applicator comprises a porous material. The porous material may be a sponge with an open cell structure.

In certain embodiments, the applicator has a perimeter which does not extend beyond a perimeter of the backing.

In certain embodiments, the backing is more rigid than the applicator.

In certain embodiments, the applicator has a wedge-shaped configuration.

In certain embodiments, the porous material has rounded edges.

In certain embodiments, the applicator is attached to the backing by an adhesive. The adhesive may comprise an adhesive composition which is not reactive to the anti-fog solution stored in the container portion. The adhesive composition may comprise a natural rubber.

In certain embodiments, the seal comprises a frangible wall separating the container portion from the applicator portion.

In certain embodiments, the container portion is a frangible ampoule, the device further comprising a handle, the ampoule being disposed within the handle.

In certain embodiments, the seal comprises a seal layer attached to an open ended tip of the container portion.

In certain embodiments, the seal comprises a frangible tip of the container portion.

In certain embodiments, the tip has a duck-bill configuration.

### Definitions

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example “A and/or B” is to be taken as specific disclosure of each of (i) A, (ii) B and (iii) A and B, just as if each is set out individually herein.

### BRIEF DESCRIPTION OF DRAWINGS

Further aspects and advantages of the present invention will become better understood with reference to the description in association with the following in which:

FIG. 1 is a perspective view from the front of a dispensing device, according to certain embodiments of the present technology;

FIG. 2 is a cross-sectional view through the line 2-2′ of FIG. 1, according to certain embodiments of the present technology;

FIG. 3 is a perspective view from the front of a dispensing device, according to certain other embodiments of the present technology;

FIG. 4 is a cross-sectional view through the line 4-4′ of FIG. 3, according to certain embodiments of the present technology;

FIG. 5 is a perspective view from the front of a dispensing device, according to certain other embodiments of the present technology;

FIG. 6 is a cross-sectional view through the line 6-6′ of FIG. 3, according to certain embodiments of the present technology;

FIG. 7 is a close-up view of a port between an applicator portion and a container portion of the dispensing device of FIGS. 5 and 6;

FIG. 8 is a perspective view from the front of a portion of a dispensing device, according to certain other embodiments of the present technology; and

FIG. 9 is a perspective view from the front of a portion of a dispensing device, according to certain other embodiments of the present technology.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

Broadly, there is provided a dispensing device 10 for dispensing anti-fog solution to a medical device (not shown). The dispensing device is a single-piece unit which can dispense liquid without requiring manipulation by two hands of a user. In non-limiting examples, the medical device is an endoscope, the anti-fog solution being applied to a lens of the endoscope to maintain or improve visibility through the lens. In other examples, the medical device is any other type of device having a surface for which avoidance of visual impairment such as fogging or blurring through condensation or the like is desired such as a mirror or a camera.

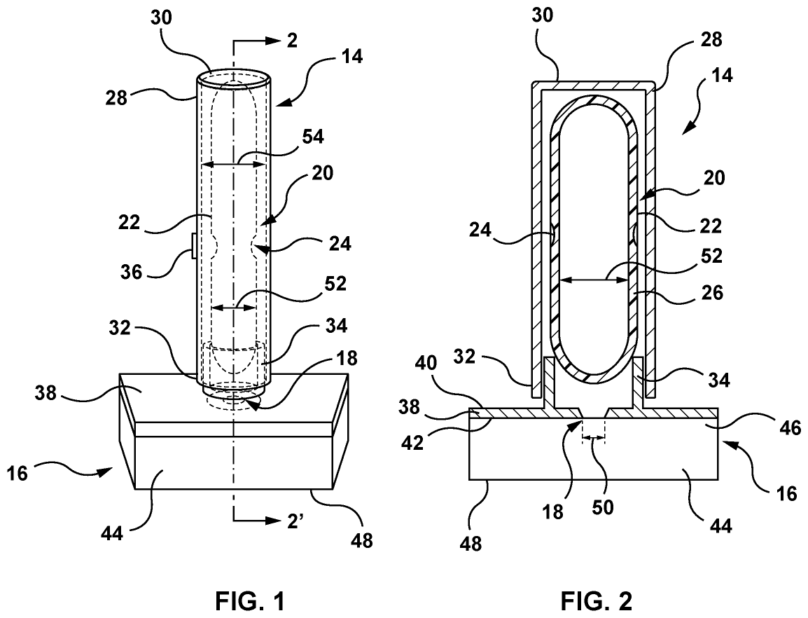

Referring initially to FIGS. 1 and 2, there is shown a first embodiment of the dispensing device 10. The dispensing device 10 comprises a container portion 14 for storing the anti-fog solution to be dispensed; and an applicator portion 16, fluidly connectable to the container portion 14 by a port 18. The applicator portion 16 is arranged to receive the anti-fog solution from the container portion 14 and to dispense the anti-fog solution to the surface of the medical device by contact with the medical device such as by rubbing or wiping or the like. The dispensing device 10 includes a seal which is arranged to be selectively modulated from a closed configuration in which the anti-fog solution is maintained in the container portion, and an open configuration in which the anti-fog solution can flow through the port 18 into the applicator portion 16. In the embodiment of FIGS. 1 and 2, the seal is provided by means of a frangible outer wall 22 of an ampoule 20 of the container portion 14.

The closed configuration of the ampoule 20 comprises an intact configuration of the outer wall 22 containing the anti-fog solution of the ampoule 20. Modulation to the open configuration is achieved by fracture of the outer wall 22. The ampoule 20 may be made of any suitable material such as a glass or a polymer. The glass may comprise any suitable composition such as a soda lime glass composition or a borosilicate glass composition. Borosilicate glass compositions comprise silicon dioxide and boric oxide with low levels of non-network forming oxides. Soda lime glass compositions comprise sodium oxide and calcium oxide.

In certain embodiments, the ampoule 20 has an elongate configuration with a diameter of 7.0 mm and a length of 85 mm Other dimensions are possible. The ampoule 20 is arranged to hold any volume of anti-fog solution within its available volume. In certain embodiments, the ampoule 20 is arranged to hold 6 ml of anti-fog solution. In other embodiments, the ampoule is sized and shaped to hold 5 ml, 7 ml, 8 ml, 9 ml, 10 ml, 11 ml, 12 ml, 13 ml, 14 ml, 15 ml, or 16 ml of the anti-fog solution.

The ampoule 20 is arranged to fracture on application of pressure to the ampoule to release its liquid contents. In this respect, in certain embodiments, the ampoule 20 has a fracture region 24 which is arranged to have a lower fracture stress compared to other regions of the ampoule 20. The fracture region 24 may have a wall thickness 26 that is thinner than a wall thickness of other regions, or have a weakness or fracture point introduced therein such as a score.

A handle 28 is provided attachable to the applicator portion 16 and sized and shaped to house the ampoule 20. The handle 28 has a closed distal end 30 and an open proximal end 32. The proximal end 32 is sized and shaped to engage with a spigot 34 of the applicator portion 16 to create a fluid seal therebetween. As illustrated, the connection between the spigot 34 of the applicator portion 16 and the handle 28 is a press-fit. In other embodiments, the spigot 34 and the handle 28 may be joined together by snap-fit, screw, or by any other mechanism that enables retention of the handle 28 to the applicator portion 16 during use of the dispensing device 10. The handle 28 is made from a polymeric material with sufficient deformability to enable pressure to be transferred therethrough, to the fracture region 24, to break the outer wall 22 of the ampoule 20. In certain embodiments, a visual marker 36 is provided on the handle 28 adjacent the fracture region 24 of the ampoule 20, to indicate to a user where to apply the pressure, when the ampoule 20 and the handle 28 are assembled with the applicator portion 16.

The applicator portion 16 comprises a backing 38 having a distal face 40 and a proximal face 42. The spigot 34 extends from the distal face 40. The port 18, for fluid flow from the container portion 14 to the applicator portion 16, is an opening 18 defined in the backing 38 and within a boundary defined by the spigot 34. Attached to the proximal face 42 of the backing 38 is an applicator 44 having a back surface 46 which is attached to the proximal face 42 of the backing 38 and an applicator surface 48 to which the anti-fog solution will flow for application to the medical device. The applicator 44 is a porous material such as a sponge with an open cell network. Closed cell network porous materials are also within the scope of the present technology. The porous material may have configurations other than open cell and closed cell enabling fluid flow therethrough. In certain embodiments, the porous material is a sponge made of a polyester-based material. The backing 38 is more rigid than the applicator 44 which is compressible and provides support for the applicator 44.

The applicator 44 is wedge shaped. In other embodiments, the applicator 44 has one or more of a triangular, quadrilateral, cylindrical, pad-like configuration. In certain embodiments, the applicator 44 has rounded edges. This may avoid damage to the surface of the medical device to which it is applied. In other configurations, the applicator 44 may have one or more sharp edges defining areas of pointed material which may assist in increased contact accuracy. The applicator 44 can have any suitable size or shape for the medical device surface to which it is to be applied. In certain embodiments, the applicator 44 is wedge-shaped having a height of about 10 mm, a depth of about 13 mm, and a width of about 26 mm. The applicator surface area is about 390 mm² in certain embodiments. Applicators of different dimensions are within the scope of the present technology and may be selected based on the medical device surface on which it will be used.

A perimeter of the applicator 44 is arranged to correspond to a perimeter of the backing 38 such that there is no overhang of the applicator 44. This can assist in providing an even application of the anti-fog solution to the medical device surface.

In certain embodiments, the applicator surface 48 is configured such that it has a surface tension which enables retention of a layer of the anti-fog solution thereon in use. In this respect, the applicator surface 48 may have a coating formed thereon.

In certain embodiments, the applicator 44 is attached to the backing 38 by an adhesive (not shown). The adhesive and the anti-fog solution are configured to be compatible in terms of reactivity and stability of the adhesion of the applicator 44 to the backing 38 in the presence of the anti-fog solution. In certain embodiments, the adhesive has a composition based on natural rubber which is stable in the presence of alcohol and other components of the anti-fog solution. In other embodiments, the adhesive is a polyvinyl alcohol based adhesive. In yet further embodiments, the applicator 44 is attached to the backing 38 in any other manner such as by mechanical means.

The anti-fog solution comprises a water-based or an alcohol based composition suitable for avoiding or treating condensation on the surface of the medical device. The anti-fog solution may include a surfactant. Any known anti-fog composition can be used, provided compatibility with materials of the dispensing device with which it will come into contact in terms of reactivity and stability. In certain embodiments, the anti-fog composition comprises less than 5% alcohol. In certain embodiments, the anti-fog composition comprises less than 4.5%, 4.0%, 3.5%, 3.0%, 2.5%, 2.0%, 1.5%, 1.0%, 0.5% or 0.25% alcohol.

Referring back to the port 18 formed in the backing 38, the port 18 is arranged so as to modulate a flow rate of the anti-fog solution (“fluid flow modulator”). Inventors of the present technology have determined that flow rate modulation of the anti-fog solution onto the applicator 44 is desirable for a number of reasons. Firstly, a fast flow rate will result in dripping of the anti-fog solution from the applicator 44 or the surface of the medical device into or onto the patient. Clearly this is undesirable and may cause health concerns for the patient. Secondly, too low or too high a flow rate can result in an under-saturation or an over-saturation of the porous material of the applicator 44 which can lead to smearing, spotting or other visually impairing effects on the surface of the medical device. In embodiments where the surface of the medical device is a lens, mirror or camera, this is clearly undesirable.

In this respect, the port 18 has a diameter 50, or cross-sectional area through which the fluid will flow, which is selected so as to modulate a fluid flow of the anti-fog solution.

The port diameter 50 is less than a diameter 52 of the ampoule 20, and is less than a diameter 54 of the handle 28. In certain embodiments, the port diameter 50 is about 4 mm. In certain other embodiments, the port diameter 50 is about 0.6 mm, about 0.8 mm, about 1.0 mm, about 1.2 mm, about 1.4 mm, about 1.6 mm, about 1.8 mm, about 2.0 mm, about 2.2 mm, about 2.4 mm, about 2.6 mm, about 2.8 mm, 3.0 mm, about 5.0 mm, about 3.2 mm, about 3.4 mm, about 3.6 mm, about 3.8 mm, about 4.2 mm, about 4.4 mm, about 4.6 mm, about 4.8 mm, about 5.2 mm, about 5.4 mm, about 5.6 mm, about 5.8 mm, about 3.5 mm to about 4.5 mm. In certain embodiments, the diameter of the ampoule is about 6 mm, 7 mm, 8 mm, 6.2 mm, 6.4 mm, 6.6 mm, 6.8 mm, 7.2 mm, 7.4 mm, 7.6 mm, 7.8 mm, 8.2 mm, 8.4 mm, 8.6 mm, 8.8 mm, about 6.5 mm to about 7.5 mm.

In certain embodiments, a cross-sectional surface area of the port 18 is less than a cross-sectional surface area of the ampoule 20, and less than a cross-sectional surface area of the handle 28. In certain embodiments, the cross-sectional surface area of the port 18 is about 12.6 mm².

In certain other embodiments, the cross-sectional surface area of the port 18 is between about 0.3 mm² for a 0.6 mm port up to about 26.4 mm² for a 5.8 mm port. In certain embodiments, the cross-sectional surface area of the port 18 is between about 7.1 mm² and about 19.6 mm². In certain embodiments, the cross-sectional surface area of the port 18 is about 5.1 mm², 6.6 mm², 7.1 mm², 9.0 mm², 9.6 mm², 12.6 mm², and 15.9 mm². In certain other embodiments, the cross sectional area for the ampoule 20 ranges from between about 28.3 mm² for a 6 mm ampoule up to about 60.8 mm² for a 8.8 mm ampoule. A person skilled in the art may select the port diameter 50 based on a viscosity of the liquid to be dispensed.

Inventors have discovered that a surface area reduction factor between the port 18 and the container portion 14 provides an effective fluid flow modulation in certain embodiments. Specifically, a surface area reduction factor (cross-sectional surface area of the ampoule divided by cross-sectional surface area of the port) of about 1.1 (for a 6 mm ampoule to a 5.8 mm port) to about 14.6 (for a 8.8 mm ampoule to a 0.6 mm port), or about 3 to about 13 provides a satisfactory anti-fog solution fluid flow in terms of efficacy of application to the medical device surface and safety to the patient. Table 1 illustrates surface area reduction factors according to the present technology.

TABLE 1
Surface area reduction factors of embodiments of
the dispensing device of the present technology.

| | Cross sectional | |
|---|---|---|
| Cross-sectional | surface area of | Surface area |
| surface area of | the container | reduction |
| the port/mm | portion/mm | factor |
| 15.9 | 50 | 3.1 |
| 12.6 | 50 | 4.0 |
| 9.6 | 50 | 5.2 |
| 7.1 | 50 | 7.0 |
| 5.1 | 63.6 | 12.5 |
| 6.6 | 63.6 | 9.6 |
| 9.0 | 63.6 | 7.1 |

In certain embodiments, the surface area reduction factor also takes into account a volume of the anti-fog solution contained in the ampoule 20.

Inventors have also discovered that a volume ratio of the anti-fog solution in the container portion 14 relative to a volume of the applicator 44 plays a role in the fluid flow modulation. In this respect, a fluid flow modulator of the dispensing device 10 can be said to comprise volume ratio between the volume of the anti-fog solution in the container portion 14 relative to a volume of the applicator 44. In certain embodiments, the volume ratio is about 3380 mm³/6 ml (563 mm³/ml). In certain other embodiments, a range of the volume ratio is between about 400 mm³/ml and about 1000 mm³/ml.

In use, pressure is applied to the handle 28 which in turn breaks the seal (ampoule outer wall 22) to release the anti-fog solution into the handle 28. The anti-fog solution will flow through the port 18, with a controlled fluid flow, and into contact with the applicator 44 and the applicator surface 48 from where the anti-fog solution can be applied to the medical device surface. Applying further pressure on the applicator 44 with the seal 22 in the open configuration can further control the fluid flow rate and supply the anti-fog solution to the medical device surface.

The dispensing device is a single use device and is sterilizable by gamma irradiation. The dispensing device 10, in use, is a unitary piece which reduces a risk of parts of the device becoming lost or forgotten in a body cavity of the patient whilst also delivering effective volumes and flow rates of anti-fog solution to the medical device surface in certain embodiments.

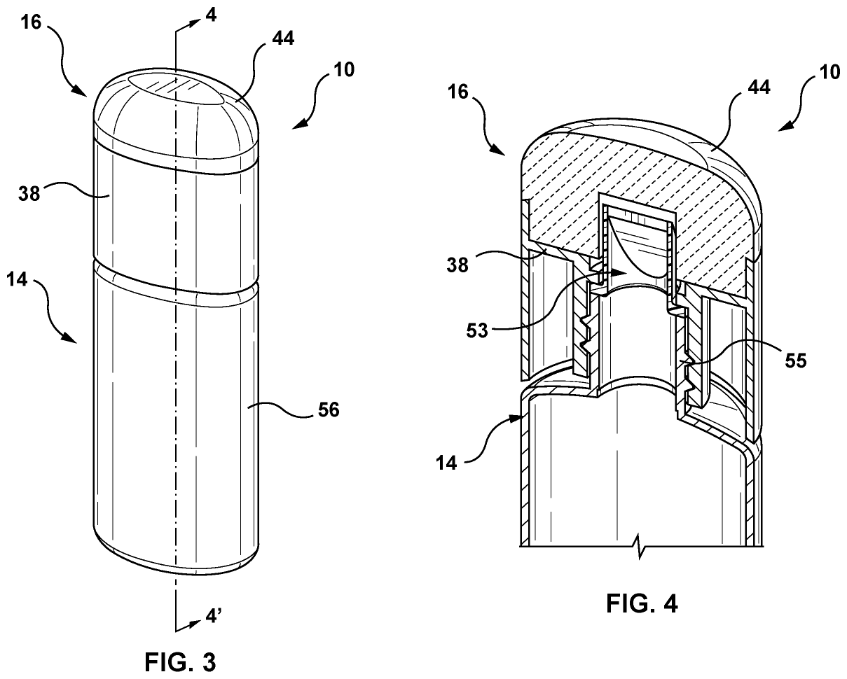

The embodiment of FIGS. 3 and 4 differs from those of FIGS. 1 and 2 in that the container portion 14 comprises a bottle 56 containing the anti-fog solution and including a tip 53 configurable between the closed configuration and the open configuration. There is no ampoule in this embodiment. The tip 53 has a duck-bill configuration. The tip 53 may comprise a one-piece construction and having a sealed end which is arranged to frangibly open. Alternatively, the tip 53 may be have an open end which is sealed by a seal layer, such as propylene or other polymer, and which is arranged to break or detach from the tip 53 when pressure is applied. The application of pressure for modulation from the closed configuration to the open configuration may include a downwards force onto the applicator 44. In this embodiment, the backing 38 is arranged to connect to a neck 55 of the bottle 56. The applicator 44 is a cylindrical shape.

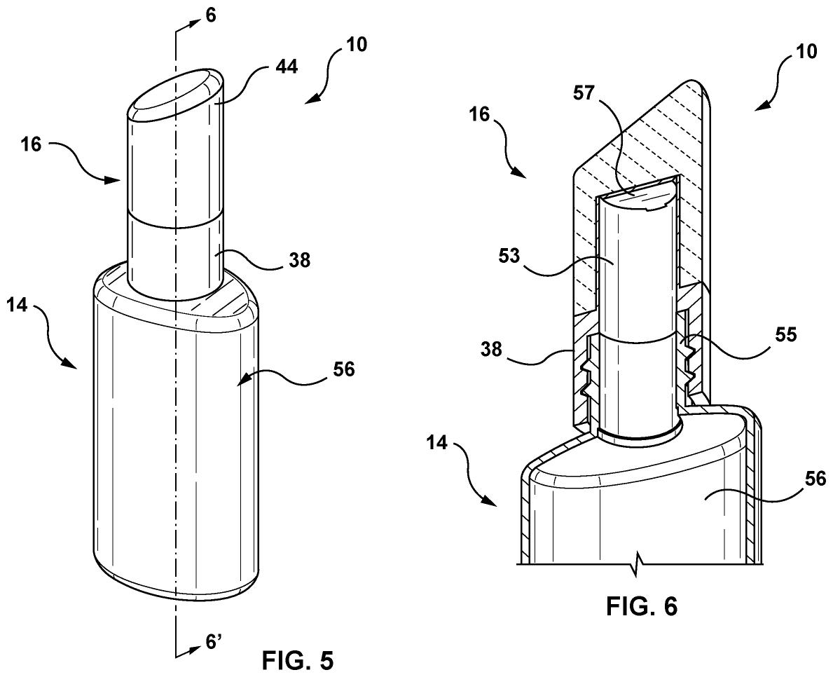

The embodiment of FIG. 5 differs from that of FIGS. 3 and 4 in that the shape of the applicator 44 is wedge-shaped.

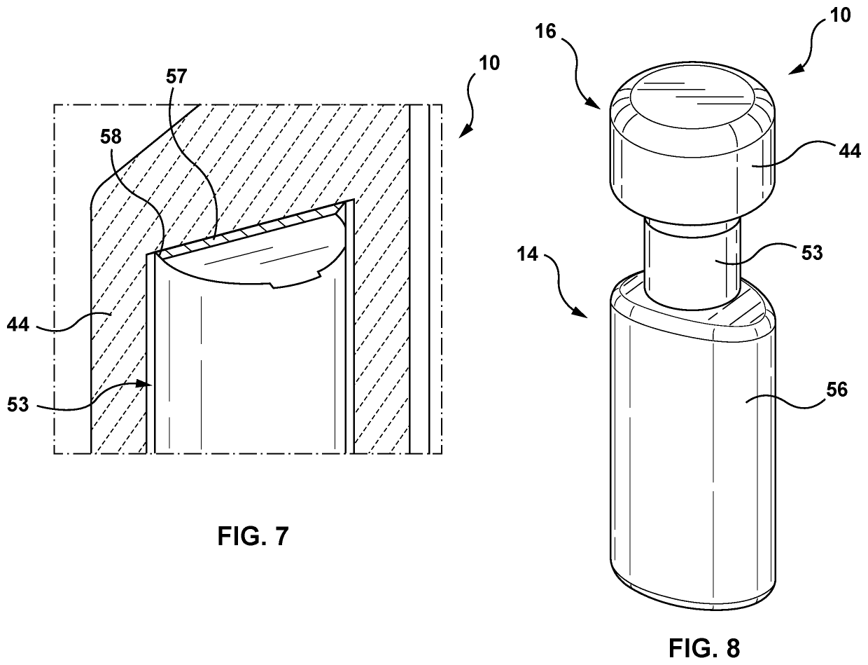

The embodiment of FIGS. 6 and 7 differ from that of FIGS. 3-5 in that the tip 53 and the backing 38 are one-piece, and that the tip is not duck-bill shaped. The tip 53 has an open end which is sealed by the seal layer 57. The seal layer 57 is attached to a top edge 58 of the tip 53 and is arranged to detach from the top edge 58 at a point of weakness.

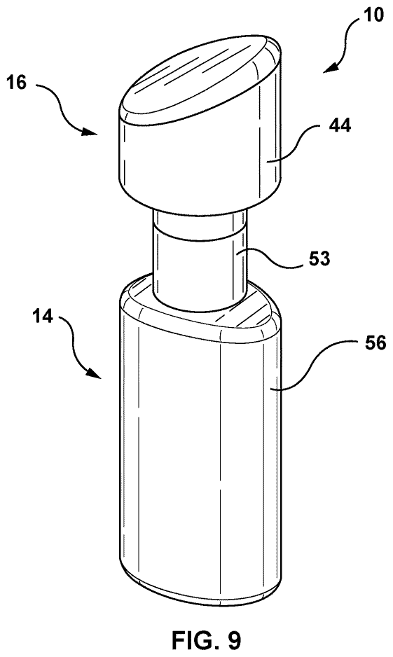

The embodiments of FIGS. 8 and 9 differ from that of other embodiments in that the applicator 44 has a wider diameter than the bottle neck 55 and the tip 53. In the embodiment of FIG. 8 the applicator is cylindrical shaped, and in the embodiment of FIG. 9, the applicator is wedge-shaped.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the invention is not limited to the particular embodiments described and illustrated herein but includes all modifications and variations falling within the scope of the invention as defined in the appended claims.

1. A dispensing device for dispensing anti-fog solution to a surface of medical device, the dispensing device comprising:
a container portion for storing the anti-fog solution to be dispensed;
an applicator portion comprising a backing and an applicator attached to the backing and defining a port within the backing for fluid connection between the container portion and the applicator;
a seal which is arranged to be selectively modulated from a closed configuration in which the anti-fog solution is maintained in the container portion, and an open configuration in which the anti-fog solution can flow through the port into the applicator portion; and
a fluid flow modulator for controlling the flow of the anti-fog solution from the container portion to the applicator portion.

2. The dispensing device of claim 1, wherein the fluid flow modulator is a surface area reduction factor at the port compared to the container portion of about 1.1 to about 14.6, or about 3 to about 13, preferably about 4.

3. The dispensing device of claim 1 or claim 2, wherein the port has cross-sectional surface area of about 2 mm² and the container portion has a cross-sectional surface area of about 12.6 mm².

4. The dispensing device of any of claims 1 to 3, wherein the applicator surface area is about 390 mm².

5. The dispensing device of any of claims 1-4, wherein the volume of anti-fog solution in the container option is about 6 ml.

6. The dispensing device of any of claims 1-5, wherein the fluid flow modulator comprises a volume ratio of volume of the anti-fog solution in the container portion to a volume of the applicator of between about 400 mm³/ml and about 1000 mm³/ml.

7. The dispensing device of any one of claims 1-6, wherein the seal is arranged to be modulated from the closed configuration to the open configuration by application of a pressure to one or both of the container portion and the applicator portion.

8. The dispensing device of any one of claims 1-7, wherein the seal comprises a material which does not react with the anti-fog solution when in the closed position.

9. The dispensing device of any one of claims 1-8, wherein the applicator comprises a porous material.

10. The dispensing device of claim 9, wherein the porous material is a sponge with an open cell structure.

11. The dispensing device of any one of claims 1-10, wherein the applicator has a perimeter which does not extend beyond a perimeter of the backing.

12. The dispensing device of claim 11, wherein the backing is more rigid than the applicator.

13. The dispensing device of any one of claims 1-12, wherein the applicator has a wedge-shaped configuration.

14. The dispensing device of any one of claims 1-13, wherein the porous material has rounded edges.

15. The dispensing device of any one of claims 1-14, wherein the applicator is attached to the backing by an adhesive.

16. The dispensing device of claim 15, wherein the adhesive comprises an adhesive composition which is not reactive to the anti-fog solution stored in the container portion.

17. The dispensing device of claim 16, wherein the adhesive composition comprises a natural rubber.

18. The dispensing device of any one of claims 1-17, wherein the seal comprises a frangible wall separating the container portion from the applicator portion.

19. The dispensing device of claim 18, wherein the container portion is a frangible ampoule, the device further comprising a handle, the ampoule being disposed within the handle.

20. The dispensing device of any one of claims 1-17, wherein the seal comprises a seal layer attached to an open ended tip of the container portion.

21. The dispensing device of any one of claims 1-17, wherein the seal comprises a frangible tip of the container portion.

22. The dispensing device of claim 20 or claim 21, wherein the tip has a duck-bill configuration.
